(19) United States
(12) Patent Application Publication
(10) Pub. No.: US 2026/0253219 A1
(43) Pub. Date: Aug. 27, 2026

(54) Method for Positioning a Patient, Processing Device, Magnetic Resonance Apparatus, Computer Program, and Data Storage Medium

(71) Applicant: Siemens Healthineers AG, Forchheim (DE)

(72) Inventor: Fabian Wagner, Erlangen (DE)

(73) Assignee: Siemens Healthineers AG, Forchheim (DE)

(21) Appl. No.: 19/545,072

(22) Filed: Feb. 20, 2026

(30) Foreign Application Priority Data
Feb. 21, 2025 (EP) 25159429.7

(51) Int. Cl. G06T 7/00; A61B 5/00; A61B 5/055; G06N 3/08; G06T 7/11; G06T 7/73; G06V 10/764; G06V 20/70; G16H 40/63

(52) U.S. Cl. CPC G06T 7/0012; A61B 5/055; A61B 5/704; G06N 3/08; G06T 7/11; G06T 7/74; G06V 10/764; G06V 20/70; G16H 40/63; G06T 2200/24; G06T 2207/20081; G06T 2207/20084; G06T 2207/30008; G06V 2201/033

(57) ABSTRACT

The disclosure is directed to a computer-implemented method for positioning a patient with respect to at least one component of a medical imaging apparatus for capturing result image data. The method may include capturing preliminary image data, which at least partially depicts at least one specified anatomical feature of the patient while the patient, or a segment of the patient that comprises the anatomical feature, is supported by a support apparatus of the medical imaging apparatus; ascertaining a feature position of the specified anatomical feature in the preliminary image data; and always, or on fulfillment of a trigger condition that depends on the feature position, actuating an actuator of the imaging apparatus to move the support apparatus into a target position ascertained based on the feature position and/or outputting a notification relating to the target position to a user of the imaging apparatus.

### CROSS REFERENCE TO RELATED APPLICATIONS

This patent application claims priority to European Patent Application No. 25159429.7, filed Feb. 21, 2025, which is incorporated herein by reference in its entirety.

### BACKGROUND

The disclosure relates to a computer-implemented method for positioning a patient with respect to at least one component of a medical imaging apparatus for capturing result image data. The disclosure also relates to a computer-implemented method for providing a model trained by machine learning, to a processing device, to a magnetic resonance apparatus, to a computer program, and to a data storage medium.

Magnetic resonance tomography (MRT) is a non-invasive imaging method which allows visualization of the internal anatomy and tissue in the human body. In particular, based on the measurement data, volumetric 3D images can be reconstructed, which can be used as the basis for diagnostic decisions in the clinical workflow. MRT acquisitions are normally planned by a doctor, who specifies the anatomical region to be examined. The scan is then typically performed by medical professionals. In this process, the patient is first positioned such that the desired anatomical region can be depicted. This is normally done using easily discernible landmarks such as joints or large anatomical features for rough alignment of the patient. In order to capture the position of the anatomy of interest accurately within the field of view (FOV) of the magnetic resonance apparatus, a rapid low-resolution localizer scan with a large field of view is carried out.

The positioning of the patient can be hampered if the exact position of relevant landmarks cannot be discerned by the medical professionals. For example, when imaging at the knee, a flexible local coil placed around the knee is often used, which obscures the knee joint. For this and other reasons, it may happen that the patient is positioned incorrectly, with the result that in some circumstances the anatomical region to be examined is not fully depicted in the localizer scan, or at least is far away from a target position, for instance from the isocenter of the imaging.

It can therefore be necessary after carrying out the localizer scan to correct the patient position by manually adjusting the patient couch. For example, when there is a large deviation in the actual position of the patient from a target position, it is often not possible for the medical professionals to make an immediate correct estimate of the required position correction, with the result that in daily clinical routine, the position adjustment is performed in many cases iteratively based on the principle of trial and error.

Such a procedure is time-consuming and labor-intensive, however, and therefore leads to less efficient use of the magnetic resonance apparatus and an increased burden on medical professionals. In addition, achieving precise positioning, which is typically necessary to achieving a high image quality, within a reasonable amount of time requires a considerable amount of training and experience on the part of the medical professionals, which can result in high training costs and long familiarization times.

### BRIEF DESCRIPTION OF THE DRAWINGS/FIGURES

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

FIG. 1 shows a magnetic resonance apparatus including a processing apparatus according to an exemplary embodiment of the disclosure.

FIG. 2 is a flowchart of a method for positioning a patient according to an exemplary embodiment of the disclosure.

FIG. 3 shows an example of preliminary image data evaluated in the method in accordance with FIG. 2.

FIG. 4 is a flowchart of method for providing a model trained by machine learning according to an exemplary embodiment of the disclosure.

FIG. 5 shows an example of a structure of a model trained, or trainable, by machine learning, according to an exemplary embodiment of the disclosure, which can be used in the exemplary embodiments of FIG. 2 and FIG. 4 as the segmentation algorithm or as the model to be trained.

The exemplary embodiments of the present disclosure will be described with reference to the accompanying drawings. Elements, features and components that are identical, functionally identical and have the same effect are-insofar as is not stated otherwise respectively provided with the same reference character.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring embodiments of the disclosure. The connections shown in the figures between functional units or other elements can also be implemented as indirect connections, wherein a connection can be wireless or wired. Functional units can be implemented as hardware, software or a combination of hardware and software.

Therefore the object of the disclosure is to define an improved approach for positioning a patient with respect to a medical imaging apparatus.

The object is achieved according to the disclosure by a method for positioning a patient with respect to at least one component of a medical imaging apparatus for capturing result image data, which may comprise the following steps:
capturing preliminary image data, which at least partially depicts at least one specified anatomical feature of the patient while the patient, or a segment of the patient that comprises the anatomical feature, is supported by a support apparatus of the medical imaging apparatus;
ascertaining a feature position of the specified anatomical feature in the preliminary image data; and always, or on fulfillment of a trigger condition that depends on the feature position,
actuating an actuator of the imaging apparatus to move the support apparatus into a target position ascertained based on the feature position and/or outputting a notification relating to the target position to a user of the imaging apparatus.

It was found as part of the disclosure that for positioning the patient, relevant anatomical features can be localized in an automated manner in preliminary image data, which in medical imaging modalities is often captured anyway before the main imaging, for instance as the localizer scan for a magnetic resonance acquisition as mentioned in the introductory part, typically with sufficient accuracy to ascertain a deviation of their feature position from a target feature position desired for the main imaging. With regard to positioning the patient, it can also be assumed in many usage cases, at least approximately, that the relevant anatomical features can be presumed to be spatially fixed with respect to the support apparatus. Hence the target position of the support apparatus can be ascertained, for example, as a relative target position in relation to an actual position of the support apparatus during capture of the preliminary image data, which relative target position corresponds to a deviation of the feature position from a specified target feature position. In medical imaging apparatuses, however, an absolute position of the support apparatus while the preliminary image data is being ascertained is often also known, and therefore the target position can also be determined as an absolute position.

In principle, it is possible that the target position fully defines the location and/or the orientation of the support apparatus in absolute terms or relative to the situation during capture of the preliminary image data, such as with respect to all three degrees of freedom of movement and/or rotation. Depending on the degrees of freedom of movement by the actuator or by the support apparatus, and/or depending on which type of anatomical feature is being viewed, it can be expedient, however, for the target position to specify only an absolute position or relative position for one or two degrees of freedom of movement and/or rotation. For example, a patient couch used as the support apparatus may be capable of displacement solely in the longitudinal direction and/or one transverse direction.

The notification relating to the target position can be used to assist a user in manual adjustment of the support apparatus. For example, the notification can prompt the user to displace the support apparatus a specified distance of travel away in a direction indicated in the notification, and/or to tilt it through a specified angle about a specified axis.

In an exemplary embodiment, the trigger condition can be fulfilled if, or can only be fulfillable when, the deviation of the ascertained feature position from a specified actual feature position exceeds a specified limit value. In the simplest case, this can take into account solely the magnitude of the deviation, although it is also possible to specify different limit values for different directions of the deviations, or the like.

It is possible that solely the actuation of the actuator takes place solely on fulfillment of the trigger condition, whereas the issuing of the notification relating to the trigger condition always takes place, or takes place already on fulfillment of a sub-condition of the trigger condition, or even when the trigger condition is not fulfilled. For example, the notification relating to the target position that is output when the trigger condition is not fulfilled can be a notification that the patient or the support apparatus is already positioned correctly or at the target position.

The component with respect to which the patient is meant to be positioned can be a component involved directly in the imaging or in the capture of data on which the imaging is based. If the imaging apparatus used is a magnetic resonance apparatus, the positioning can be performed with respect to at least one receive coil and/or with respect to at least one transmit coil and/or with respect to at least one field coil. In standard magnetic resonance apparatuses, said coils are typically at least in part spatially fixed with respect to an open space for the patient, and therefore the positioning can be performed with respect to this open space. In the case of X-ray imaging, on the other hand, the positioning can be performed with respect to an X-ray tube and/or an X-ray detector. For other imaging modalities, for instance for ultrasound imaging and/or for molecular imaging, the positioning can be performed with respect to each at least one sensor used in capturing the image data.

The support apparatus can be a patient couch, although it can also be, for example, a seat or a resting surface for the patient's limb comprising the anatomical feature. The position of the patient and of the support apparatus and the feature position can describe a location and/or an orientation of the patient or of the support apparatus or of the feature respectively.

A segmentation algorithm can perform segmentation of at least one bone of the patient in the preliminary image data, wherein the feature position is ascertained based on the segmentation. In particular, large bone structures are well suited to defining a positioning of the patient with respect to an imaging region and hence with respect to the imaging-related component of the imaging apparatus. Similar to the case in manual positioning, for example, the focus here can be on positioning at least one joint of the patient in the imaging region, allowing empirical values from the hitherto usual manual patient positioning to be used to specify the target feature position.

Segmentation of at least one bone as the anatomical feature or for defining the anatomical feature is also expedient in magnetic resonance imaging even though standard magnetic resonance sequences are not optimized for high contrasts in the depiction of bone. Typically, at least for preparatory localizer scans, measurement sequences are used that provide sufficiently high bone contrasts, for instance because the positions of joints, as already explained in the introductory part, are also highly relevant in manual positioning of the patient and also when planning slices to be scanned as part of the magnetic resonance imaging.

The bone or the segment depicting the bone can be used directly as the anatomical feature, the feature position of which is being determined. However, since larger bones, for instance the fibula and tibia in imaging a knee, are often not fully depicted in preliminary image data typically used for localization, it can be expedient to use as the anatomical feature a certain part of the bone, so for instance the proximal end or distal end of the bone.

It has proved particularly expedient, however, to localize gaps left between bones or boundary surfaces between bones as the anatomical feature. Thus for two of the bones that are adjacent to each other respective segments in the preliminary image data can be determined, each segment depicting the associated bone, wherein an intermediate region situated between these segments, or a boundary line or boundary surface between these segments, is used as the anatomical feature, the feature position of which is being determined.

The segmentation algorithm used can be a model trained by machine learning. In the field of segmentation of anatomical features or of medical image data, machine-learning algorithms allow particularly robust segmentation, and robust assignment of segments to specific anatomical features, so in the method according to the disclosure to bones. Thus robust segmentation and classification is possible even when the segmented anatomical features are depicted only partially and/or when the image quality, so for example an image resolution and/or a signal-to-noise ratio, of the preliminary image data is relatively low, for instance if a localizer scan with a short scan time is used.

The model trained by machine learning can process (e.g., directly) the preliminary image data as input data. Alternatively, however, it is also possible to pre-process the preliminary image data to provide the input data. The training of the model may be performed outside the claimed method and can be carried out, for example, at another location and/or by other people than the method for positioning the patient. For example, the training can be performed by a manufacturer of the medical imaging apparatus, whereas the method for positioning the patient is applied at the premises of an end user, so for instance in a hospital or a medical practice.

In general, a model trained by machine learning models cognitive functions that humans associate with other human brains. As a result of training based on training data (machine learning), the trained function is capable of adapting to new circumstances and detecting and extrapolating patterns. Another term for “a model trained by machine learning” is “a trained function”.

Generally speaking, parameters of a trained model can be adapted by training. In particular, supervised learning, semi-supervised learning, unsupervised learning, reinforcement learning and/or active learning can be used. Furthermore, representation learning (also known as feature learning) can also be employed. The parameters of the trained function can be adapted iteratively by a plurality of training steps. In particular, a specific cost function can be minimized in the training. For example, the backpropagation algorithm can be employed in the training of a neural network.

A trained function may comprise, for example, a neural network, a transformer, a support vector machine (SVM), a decision tree and/or a Bayes network, and/or the trained function can be based on k-means clustering, Q-learning, genetic algorithms and/or association rules. In particular, a neural network may be a deep neural network, a convolutional neural network (CNN) or a deep CNN. In addition, the neural network can be an adversarial network, a deep adversarial network, and/or a generative adversarial network (GAN).

The model trained by machine learning can be or comprise a neural network having a U-Net structure. A neural network having a U-Net structure has an encoder-decoder structure, which is particularly well suited to segmentation even of fine structures. The fundamental network architecture was first described in O. Ronneberger et al., “U-Net: Convolutional Networks for Biomedical Image Segmentation”, arXiv: 1505. 04597v1. For example, the open-source nnU-Net can be used (https://github.com/MIC-DKFZ/nnUNet, see also Isensee, F., et al. nnU-Net: a self-configuring method for deep learning-based biomedical image segmentation. Nat Methods 18, 203-211 (2021). https://doi.org/10.1038/s41592-020-01008-z).

The trained model can be provided, for example, by supervised training based on the nnU-Net or another model to be trained. Suitable training datasets that can be used are, for example, training image data that is segmented and classified manually, for instance, in order to assign to the individual image points of the respective training image data a class that classifies the anatomical feature depicted there.

In order to avoid an unexpected movement of the support apparatus and hence of the patient, it can be expedient to perform the actuation of the actuator only after approval by the user. It is therefore possible that on fulfillment of a first sub-condition of the trigger condition, which first sub-condition evaluates the target position, an approval request is first output to the user, wherein the actuation of the actuator to move the support apparatus into the target position only takes place on fulfillment of a second sub-condition of the trigger condition, wherein the second sub-condition is fulfilled if, or can only be fulfilled when, an operating input by the user indicating approval of the movement is captured at the medical imaging apparatus and/or at an approval apparatus in communication with the medical imaging apparatus.

The approval request can be or comprise the notification relating to the target position. For example, this can also allow the user to choose by way of different operating inputs between different options, for instance between automatic positioning of the support apparatus and/or adjustment of the support apparatus performed manually or by means of actuators controlled by user inputs and/or positioning of the patient on the support apparatus with subsequent repositioning of the support apparatus.

The notification relating to the target position and/or the approval request may comprise explanatory information for the user as to why a movement of the support apparatus appears appropriate. The explanatory information can be a text or a voice message, for example. In an exemplary embodiment, the explanatory information can be fixed for the medical imaging apparatus or for a particular imaging task, for example for depicting a particular anatomical feature.

In an exemplary embodiment, however, one of a plurality of specified pieces of explanatory information can be selected always or on fulfillment of the trigger condition and/or the first sub-condition depending on the feature position, and output as part of the notification relating to the target position and/or of the approval request.

The particular explanatory information can be or comprise a reason for why a change in the position of the support apparatus and hence of the patient is appropriate for the current imaging task. By taking into account the feature position in the selection of the explanatory information, it is possible to distinguish between, for example, cases in which, in the current position of the patient, an anatomical feature that is typically relevant in the current imaging task will be unnecessarily far away from an isocenter of the imaging, which may reduce the image quality, and cases for which, for example, one of a plurality of potentially relevant anatomical features cannot be fully depicted in some circumstances. This can allow the user to judge himself whether the stated reason is or is not relevant in his opinion to the actual imaging task in hand. On this basis, for example, the user can make a decision about whether further time shall be spent on repositioning or whether imaging shall take place immediately.

The assignment of the explanatory information to the feature positions can be made, for instance, by a lookup table or the like. For example, as part of providing a program implementing the method, one or more experts can assign different explanatory information to different values or value ranges of the feature position and/or values or value ranges of individual coordinates of the feature position.

After the actuation of the actuator to move the support apparatus into the target position, the capture of the preliminary image data can be repeated automatically in order to determine new preliminary image data. Based on the new preliminary image data, a new feature position of the specified anatomical feature, for example, can then be ascertained, for instance in order to validate that this now coincides with a target feature position or that a deviation from the target feature position now lies below a specified limit value. If this is not the case, then a new target position can be ascertained based on the new feature position, for example, and used to move the support apparatus again by means of the actuator, and/or a notification relating to the incorrect positioning can be output to the user.

Additionally, or alternatively, the new preliminary image data can be used for planning the main imaging, which is used to capture the result image data. For example, when a magnetic resonance apparatus is employed as the medical imaging apparatus, planning of imaging slices can be based on the new preliminary image data.

The medical imaging apparatus used can be a magnetic resonance apparatus. As already explained in the introductory part, the positioning of a patient for magnetic resonance imaging can be hampered additionally by a local coil obscuring at least one relevant anatomical feature, for instance a joint. Therefore, the method according to the disclosure is particularly relevant in this imaging modality. In addition, in the case of magnetic resonance imaging, a localizer scan is often used anyway to validate the patient position and/or for slice planning, so that the procedure according to the disclosure can be integrated easily into routine clinical practice or into the standard imaging procedure, allowing a significant reduction in the amount of time and effort involved in capturing image data, as already explained.

The disclosure also relates to a computer-implemented method for providing a model trained by machine learning, which is configured to be used in the computer-implemented method according to the disclosure for positioning a patient as the segmentation algorithm and/or for ascertaining the feature position, comprising the following steps:
receiving input training data;
receiving output training data, wherein the output training data specifies a class for each of the pixels or voxels in the input training data and/or describes a segmentation of the input training data;
training the model by machine learning based on the input training data and the output training data; and
   providing the model trained by machine learning.

In particular, at least one specified bone is segmented in the input training data, or a group of pixels or voxels is classified as the specified bone. In particular, at least two bones are each segmented or classified.

As already explained above, it is hence possible to use supervised learning, known per se, in which a cost function can be minimized that depends on a difference measure between the output data ascertained based on the input training data by the model and the output training data. The parameters of the trained model can then be adapted iteratively in order to minimize the cost function for a given batch of training data. For example, error feedback can be used for this purpose, such as by means of the gradient descent method.

The disclosure also relates to a processing apparatus, which is configured to perform the computer-implemented method according to the disclosure for positioning a patient and/or to perform the computer-implemented method according to the disclosure for providing a model trained by machine learning. The processing apparatus can be, for example, suitably programmed data processing devices, or at least some of the aforementioned functionality can alternatively have a hard-wired implementation. The processing apparatus can be integrated in a medical imaging apparatus, or be separate therefrom. For example, it can be implemented as a workstation computer, server or Cloud solution.

The disclosure also relates to a magnetic resonance apparatus which may comprise a processing apparatus according to the disclosure. By integrating the processing apparatus according to the disclosure, and hence the implementation of the method according to the disclosure, in a magnetic resonance apparatus, the positioning of the patient can be integrated particularly seamlessly into the imaging process.

The disclosure also relates to a computer program containing instructions which are configured to perform, when executed on a data processing device, the computer-implemented method according to the disclosure for positioning a patient and/or for providing a model trained by machine learning.

The disclosure also relates to a data storage medium comprising the computer program according to the disclosure.

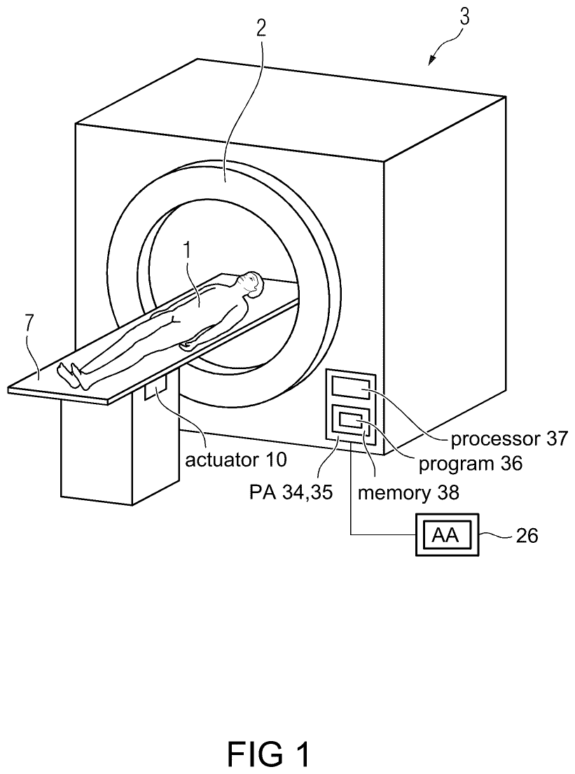

FIG. 1 shows a medical imaging apparatus 3, which in the example is a magnetic resonance apparatus and in the operating situation shown is meant to be used to capture result image data 4 relating to a patient 1.

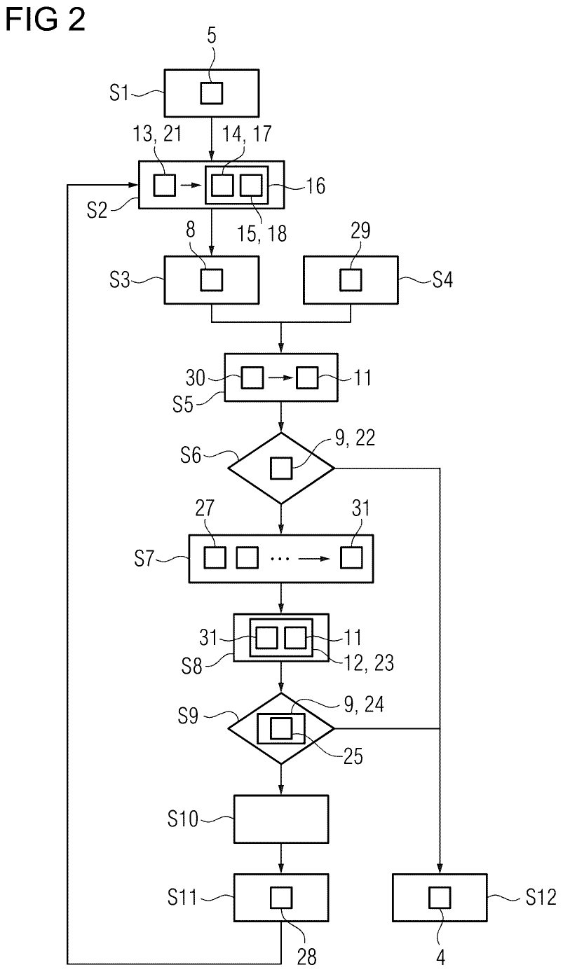

A user (not shown), so for instance a medical professional, is meant to be assisted in the positioning of the patient 1 with respect to a component 2 of the magnetic resonance apparatus 3, so for instance with respect to the patient bore. The magnetic resonance apparatus 3 may comprise for this purpose a processing apparatus 34, which is configured to determine automatically based on preliminary image data 5 of the patient 1 a target position 11 for a support apparatus 7 bearing the patient 1. In the example, after approval by the user, this target position 11 can then be set up automatically by means of the actuator 10 of the medical imaging apparatus 3. An example of a method for positioning the patient 1 that is used for this purpose will be explained later in greater detail with reference to FIG. 2.

In the example, the method is implemented by a computer program 36, which is stored in a memory 38 of a data processing apparatus 35 of the medical imaging apparatus 3 and is executed by the processor 37. Additionally, or alternatively, the method may be implemented by an external processing apparatus, for instance on a workstation computer or a server. The data processing apparatus 34, 35 may include processing circuitry configured to perform one or more functions and/or operations of the data processing apparatus 34, 35. Additionally, or alternatively, one or more components of the data processing apparatus 34, 35 may include processing circuitry that is configured to perform one or more respective functions of the component(s).

In the flow diagram shown in FIG. 2 of the method for positioning the patient 1, first, in step S1, preliminary image data 5 is captured, which at least partially depicts at least one specified anatomical feature 6 of the patient 1. The preliminary image data 5 can be captured for example by a localizer scan typical in the field of magnetic resonance imaging.

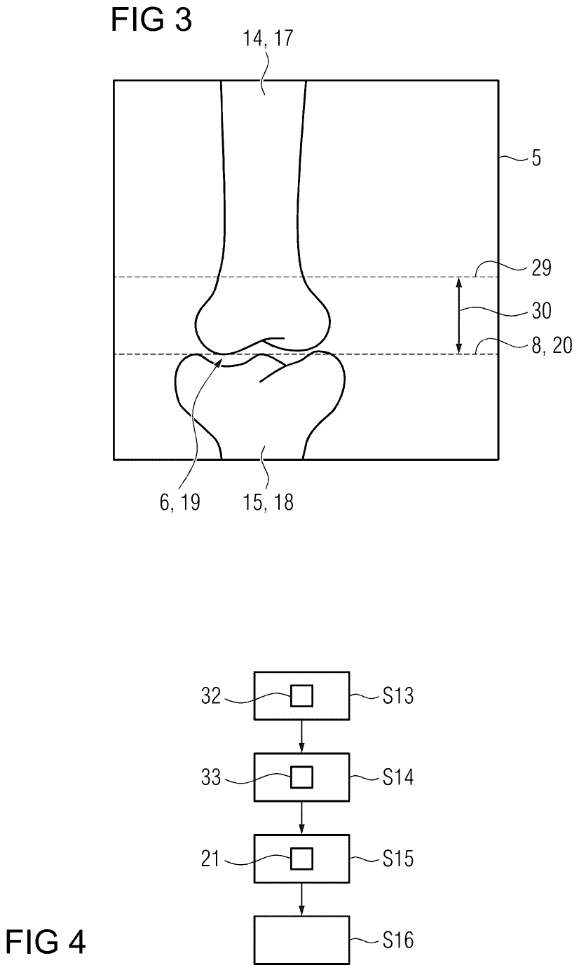

FIG. 3 shows an example of preliminary image data 5 relating to the region of a knee of the patient 1. In the example, the anatomical feature 6 viewed is an intermediate region 19 between two bones 14, 15, namely between tibia and fibula.

In step S2, the preliminary image data 5 is then segmented by a segmentation algorithm 13. In the example, the segmentation 16 is performed such that for two adjacent bones 14, 15, so in the example for the tibia and fibula, a corresponding segment 17, 18 depicting the respective bones 14, 15 is ascertained in the preliminary image data 5.

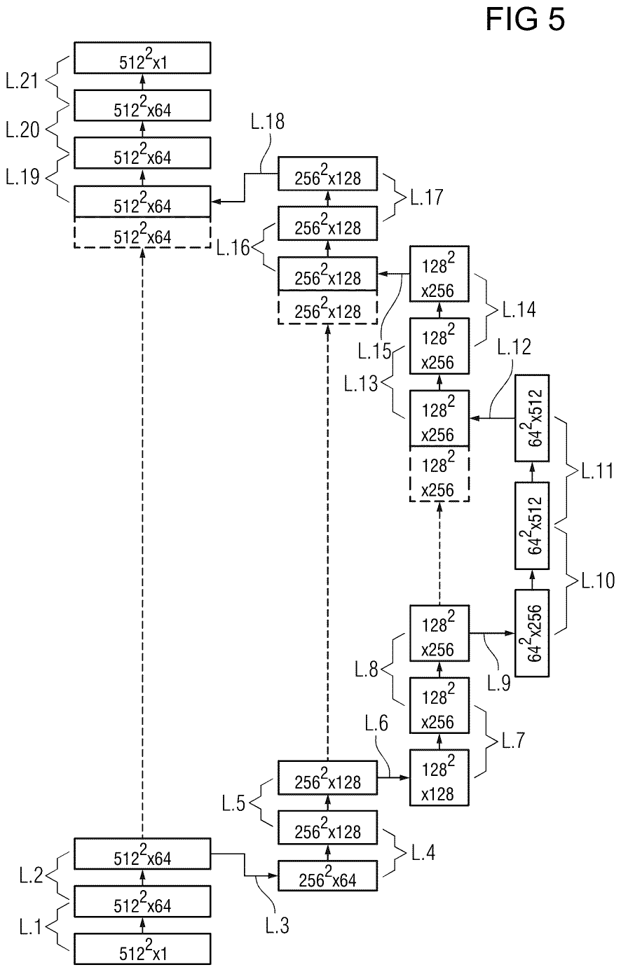

The segmentation algorithm 13 used in the example is a model trained by machine learning 21. The trained model may comprise, for example, a neural network or a cascade of a plurality of neural networks having a U-Net structure. The fundamental structure of a neural network having a U-Net structure will be explained later with reference to FIG. 5. For example, a suitably trained model based on the open-source nnU-Net already cited in the introductory part can be used. Possible training will be explained later with reference to FIG. 4.

In step S3, the feature position 8 of the anatomical feature 6 is ascertained based on the segmentation 16. In the example, it is assumed that the support apparatus 7 is meant to be displaced by the actuator 10 solely in the longitudinal direction of the patient 1, and therefore also only one target position 11 of the support apparatus 7 for this direction is meant to be determined. Thus the positions of the boundary surface 20 between the segments 17, 18 can be used as the feature position 8.

If, in an extension of the example, automatic displacement in the transverse direction of the patient 1 were also wanted, the intermediate region 19 could additionally be localized in the transverse direction in FIG. 3, or additionally or alternatively a further anatomical feature 6 could be analyzed, for example the location of a center line of one of the bones 14, 15 and hence of one of the segments 17, 18.

In step S4, a target feature position 29 is also specified. This can be specified implicitly or explicitly by the computer program 36 implementing the method, or can also be set by the user or selected automatically depending on a set imaging task. In the example, the target feature position 29 at which the intermediate region 19 or the boundary surface 20 should be situated is located in the central region in the vertical direction in FIG. 3, which corresponds to the longitudinal direction or the proximal-distal direction of the patient 1, as also shown schematically in FIG. 3.

In step S5, a target position 11 for the support apparatus 7 is then ascertained based on the deviation 30 between the ascertained feature position 8 and the target feature position 29. The target position 11 can be a relative target position of the support apparatus 7 with respect to the actual position during the capture of the preliminary image data 5. The direction in which the relative target position lies with respect to the actual positions is then obtained from whether the feature position 8 in FIG. 3 lies above or below the target feature position 29. The distance of the target position 11 from the actual position is specified by the deviation 30.

In step S6, a first sub-condition 22 of a trigger condition 9 is then checked, wherein automatic displacement of the support apparatus 7 by the actuator 10 in the example shall take place only when both the first sub-condition 22 and a second sub-condition 24 (to be discussed later) of the trigger condition 9 are fulfilled. The first sub-condition 22 is fulfilled in the example when the target position 11 deviates from the current actual position of the support apparatus 7 at least by a specified limit value.

If this is the case, then in step S7 in the example, one of a plurality of specified pieces of explanatory information 27 is first selected depending on the feature position 8 as selected explanatory information 31, which is then output to the user in step S8 as part of a notification 12 relating to the target position 11. In the example, the notification is issued via an approval apparatus 26, which is separate from the medical imaging apparatus 3 and in communication therewith, and which is shown by way of example as a touchscreen in FIG. 1. The approval apparatus 26 may include processing circuitry configured to perform one or more functions and/or operations of the approval apparatus 26. Additionally, or alternatively, one or more components of the approval apparatus 26 may include processing circuitry that is configured to perform one or more respective functions of the component(s).

The specified explanatory information 27 can give various reasons for why repositioning the patient 1 by moving the support apparatus 7 appears expedient based on the ascertained feature position 8. In the example, for which FIG. 3 shows preliminary image data 5, a knee of the patient 1 is meant to be depicted. Depending on the ascertained feature position 8, the selected explanatory information that can be output in this case is, for example, that in the current patient position, the knee probably cannot be fully depicted, or that the image quality might be impaired because of the large distance of the knee from an isocenter of the medical imaging apparatus, or that an automatic position-correction is not possible, or not appropriate, because of the very large positional error.

Since, in the example, the notification 12 may comprise the target position 11, it would be possible in principle for the user himself to correct the position of the support apparatus 11 or of the patient 1 directly based on the target position 11. Since, however, automatic position-correction by means of the actuator 10 is meant to be facilitated in the example, the notification 12 in the example acts at the same as an approval request 23, which prompts the user to actuate an operating input 25 indicating approval of the movement. In the example, this operating input 25 can be captured by means of the approval apparatus 26, so for instance also at a distance from the medical imaging apparatus 3, for example in a control room.

In step S9, it is then checked as the second sub-condition 24 of the trigger condition 9 whether such an operating input 25 has been captured. Approval by the user may not happen, for example, when the user does not want any further adjustment to the patient position, for instance to get to imaging more quickly or even to retain a specific patient positioning that is selected deliberately by the user.

If the second sub-condition 24 and thus the full trigger condition 9 is fulfilled, then, in step S10, the actuator 10 is actuated to bring the support apparatus 7 into the target position 11.

Then, in step S11, the imaging is repeated in order to capture new preliminary image data 28. The method can then be repeated from step S2 in order to validate that the feature position 8 ascertained based on the new preliminary image data 28 is sufficiently close to the target feature position 29. If this is not the case, the position of the support apparatus 7 can be readjusted, as explained above.

If, on the other hand, in step S6 the first sub-condition 22 of the trigger condition 9 is not fulfilled for the preliminary image data 5 or for the new preliminary image data 28, or if in step S9 approval for moving the support apparatus 7 is not given by the user and thus the second sub-condition 24 of the trigger condition 9 is not fulfilled, then, in step S12, the capture of the result image data 4 can proceed, wherein initially planning of this imaging can take place based on the most recently acquired preliminary image data 5 or new preliminary image data 28, for example for suitable division of the volume of interest into measurement slices.

In a development (not shown) of the described method, it might be possible, for example, in step S9 to give the user additionally the option of temporarily suspending or terminating by a certain operating input the positioning and imaging, for instance in order to reposition the patient 1 on the support apparatus 7, or the like.

A possible way of providing the segmentation algorithm 13 used in the method of FIG. 2 is explained below with reference to FIG. 4. The segmentation algorithm 13 is provided here by training a model 21 by supervised machine learning.

This is done by providing, in step S13, input training data 32, which in the example may comprise various preliminary image data that depicts for different patients the anatomical region, the segmentation of which is meant to be learned.

In step S14, output training data 33 is additionally provided, which specifies an associated target result that is meant to be obtained when the particular input training data 32 is processed by the trained model 21. Thus, the output training data 33 specifies the segmentation of the input training data 32 and can be provided, for example, by manual segmentation of the input training data 32 by experts. Such segmentation can be specified for example by assigning a class to each pixel or voxel of the input training data 32. Since, in the example described above, bones are meant to be segmented, the class can specify, for example, to which bone the particular pixel or voxel belongs, or that it does not belong to a bone.

In step S15, the model 21 is then trained by machine learning, such as iteratively. For example, error feedback based on a gradient descent method is performed here, which is known per se and in which in each iteration a cost function is evaluated for a given batch of input training data 32 and associated output training data 33. The cost function depends here on a deviation of each result of applying the model 21 in the current training state to the input training data 32 from the associated output training data 33. By ascertaining a gradient of this cost function with respect to the parameters of the model 21, it is then ascertained to what extent and in what direction the various parameters shall be adapted in the current iteration.

At the end of the training, for example after a specified number of iterations or on fulfillment of a convergence criterion, then, in step S16, the current parameterization of the model 21 can be provided as the trained model 21, so for example can be transferred from a training environment to individual delivered computer programs, processing apparatuses or medical imaging apparatuses.

An expedient approach to segmentation of image data by a model trained by machine learning is for the trained model to assign particular classes to particular pixels or voxels of the image data, and subsequently to combine pixels or voxels of the same class into a particular segment. Thus, models that implement an image-to-image transformation are suitable for this purpose.

Neural networks having a U-Net structure or cascades of such neural networks, for instance the already cited nnU-Net, are extremely well suited to image-to-image transformation and in particular, as already explained in the general part of the description, to implementing segmentation.

FIG. 5 shows an example of a U-Net structure. For the sake of simplicity, it is assumed in the example that a two-dimensional image is processed as the input data, and in turn a two-dimensional image is obtained as the result, in which the image value of the individual image points indicates, for example, whether these lie on an edge. Edges both in the x-direction and in the y-direction can be identified directly from such output data. The model shown can be trained for example by unsupervised learning using training datasets in which edges are labeled by trained personnel, for example. As part of the training, a deviation of the result from a target result can be minimized, for instance by error feedback, such as by a gradient descent method.

The input data for the trained model is a two-dimensional medical image containing 512×512 image points, with each image point containing an image value. The trained model consists of convolutional layers (identified by continuous horizontal arrows), pooling layers (identified by continuous arrows pointing downwards) and upsampling layers (identified by continuous arrows pointing upwards), with the relevant number of nodes given in the boxes. Within the U-Net structure, the input data is first downsampled (reduction in the size of the images and increase in the number of channels). Then they are upsampled (increase in the size of the images and reduction in the number of channels) in order to generate a transformed image.

All the convolutional layers L.1, L.2, L.4, L.5, L.7, L.8, L.10, L.11, L.13, L.14, L.16, L.17, L.19, L.20 apart from the last convolutional layer L.21 use a 3×3 convolution kernel with padding of 1, a ReLU activation function, and a number of filters or convolution kernels, which equals the number of channels (indicated in FIG. 5) of the respective layers. The last convolutional layer L.22 uses a 1×1 convolution kernel without padding and the ReLU activation function.

The pooling layers L.3, L.6, L.9 are max pooling layers, which replace four adjacent nodes with just one node, where the value is the maximum of the values of the four adjacent nodes. The upsampling layers L.12, L.15, L.18 are transposed convolutional layers using 3×3 kernels and stride 2, which effectively quadruple the number of nodes. The dashed horizontal arrows correspond to concatenation operations in which the output of a convolutional layer L.2, L.5, L.8 in the downsampling branch of the U-Net structure is used as an additional input for a convolutional layer L.13, L.16, L.19 in the upsampling branch of the U-Net structure. This additional input data is treated as additional channels in the input node layer for the convolutional layer L.13, L.16, L.19 in the upsampling branch.

To enable those skilled in the art to better understand the solution of the present disclosure, the technical solution in the embodiments of the present disclosure is described clearly and completely below in conjunction with the drawings in the embodiments of the present disclosure. Obviously, the embodiments described are only some, not all, of the embodiments of the present disclosure. All other embodiments obtained by those skilled in the art based on the embodiments in the present disclosure without any creative effort should fall within the scope of protection of the present disclosure.

It should be noted that the terms “first”, “second”, etc. in the description, claims and abovementioned drawings of the present disclosure are used to distinguish between similar objects, but not necessarily used to describe a specific order or sequence. It should be understood that data used in this way can be interchanged as appropriate so that the embodiments of the present disclosure described here can be implemented in an order other than those shown or described here. In addition, the terms “comprise” and “have” and any variants thereof are intended to cover non-exclusive inclusion. For example, a process, method, system, product or equipment comprising a series of steps or modules or units is not necessarily limited to those steps or modules or units which are clearly listed, but may comprise other steps or modules or units which are not clearly listed or are intrinsic to such processes, methods, products or equipment.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The exemplary embodiments described herein are provided for illustrative purposes, and are not limiting. Other exemplary embodiments are possible, and modifications may be made to the exemplary embodiments. Therefore, the specification is not meant to limit the disclosure. Rather, the scope of the disclosure is defined only in accordance with the following claims and their equivalents.

Embodiments may be implemented in hardware (e.g., circuits), firmware, software, or any combination thereof. Embodiments may also be implemented as instructions stored on a machine-readable medium, which may be read and executed by one or more processors. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact results from computing devices, processors, controllers, or other devices executing the firmware, software, routines, instructions, etc. Further, any of the implementation variations may be carried out by a general-purpose computer.

The various components described herein may be referred to as “modules,” “units,” or “devices.” Such components may be implemented via any suitable combination of hardware and/or software components as applicable and/or known to achieve their intended respective functionality. This may include mechanical and/or electrical components, processors, processing circuitry, or other suitable hardware components, in addition to or instead of those discussed herein. Such components may be configured to operate independently, or configured to execute instructions or computer programs that are stored on a suitable computer-readable medium. Regardless of the particular implementation, such modules, units, or devices, as applicable and relevant, may alternatively be referred to herein as “circuitry,” “controllers,” “processors,” or “processing circuitry,” or alternatively as noted herein.

For the purposes of this discussion, the term “processing circuitry” shall be understood to be circuit(s) or processor(s), or a combination thereof. A circuit includes an analog circuit, a digital circuit, data processing circuit, other structural electronic hardware, or a combination thereof. A processor includes a microprocessor, a digital signal processor (DSP), central processor (CPU), application-specific instruction set processor (ASIP), graphics and/or image processor, multi-core processor, or other hardware processor. The processor may be “hard-coded” with instructions to perform corresponding function(s) according to aspects described herein. Alternatively, the processor may access an internal and/or external memory to retrieve instructions stored in the memory, which when executed by the processor, perform the corresponding function(s) associated with the processor, and/or one or more functions and/or operations related to the operation of a component having the processor included therein.

In one or more of the exemplary embodiments described herein, the memory is any well-known volatile and/or non-volatile memory, including, for example, read-only memory (ROM), random access memory (RAM), flash memory, a magnetic storage media, an optical disc, erasable programmable read only memory (EPROM), and programmable read only memory (PROM). The memory can be non-removable, removable, or a combination of both.

1. A computer-implemented method for positioning a patient with respect to at least one component of a medical imaging apparatus for capturing result image data, comprising:
capturing preliminary image data that at least partially depicts at least one specified anatomical feature of the patient while the patient, or a segment of the patient that comprises the anatomical feature, is supported by a support apparatus of the medical imaging apparatus;
ascertaining a feature position of the specified anatomical feature in the preliminary image data; and
based on a trigger condition associated with the feature position: actuating an actuator of the imaging apparatus to move the support apparatus into a target position, and/or outputting a notification relating to the target position to a user of the imaging apparatus, wherein the target position is determined based on the feature position.

2. The computer-implemented method as claimed in claim 1, further comprising segmenting, based on a segmentation algorithm, at least one bone of the patient in the preliminary image data, wherein the feature position is ascertained based on the segmentation.

3. The computer-implemented method as claimed in claim 2, wherein, for two bones of the at least one bone that are adjacent to each other, determining respective segments in the preliminary image data, each segment depicting the associated bone of the two bones, wherein an intermediate region situated between the determined segments, or a boundary line or boundary surface between the determined segments, is used as the anatomical feature, the feature position of which is being determined.

4. The computer-implemented method as claimed in claim 2, wherein the segmentation algorithm is a model trained by machine learning.

5. The computer-implemented method as claimed in claim 4, wherein the model trained by machine learning is or comprises a neural network having a U-Net structure.

6. The computer-implemented method as claimed in claim 1, further comprising, based on fulfillment of a first sub-condition of the trigger condition configured to evaluate the target position, outputting an approval request to the user, wherein the actuation of the actuator to move the support apparatus into the target position only takes place on fulfillment of a second sub-condition of the trigger condition, the second sub-condition being fulfilled based on a capture of an operating input by the user indicating approval of the movement at the medical imaging apparatus and/or at an approval apparatus in communication with the medical imaging apparatus.

7. The computer-implemented method as claimed in claim 1, further comprising:
selecting one of a plurality of specified pieces of explanatory information based on the feature position and fulfillment of the trigger condition and/or a first sub-condition of the trigger condition configured to evaluate the target position; and outputting, as part of the notification relating to the target position and/or of an approval request associated with the first sub-condition.

8. The computer-implemented method as claimed in claim 1, wherein, after the actuation of the actuator to move the support apparatus into the target position, the capture of the preliminary image data is repeated automatically to determine new preliminary image data.

9. The computer-implemented method as claimed in claim 1, wherein the medical imaging apparatus is a magnetic resonance apparatus.

10. The computer-implemented method as claimed in claim 2, wherein the segmentation algorithm is a model trained by machine learning by a training method comprising:
receiving input training data;
receiving output training data, wherein the output training data specifies a class for each pixel or voxel in the input training data and/or describes a segmentation of the input training data;
training the model by machine learning based on the input training data and the output training data; and
providing the model trained by machine learning as the segmentation algorithm.

11. A processing apparatus, comprising:
one or more processors; and
memory storing instructions that, when executed by the one or more processors, cause the apparatus to perform the method of claim 1.

12. A magnetic resonance apparatus comprising the processing apparatus of claim 11.

13. At least one non-transitory computer-readable medium comprising instructions stored thereon, that when executed by one or more processors, cause the one or more processors to perform the method of claim 1.
